(19) United States
(12) Patent Application Publication
(10) Pub. No.: US 2024/0002370 A1
(43) Pub. Date: Jan. 4, 2024

(54) PYRROLE-TYPE COMPOUNDS AND USES THEREOF FOR TREATING VIRAL INFECTIONS

(71) Applicant: ENYO PHARMA, LYON (FR)

(72) Inventors: JACKY VONDERSCHER, SENOUILLAC (FR); BENOÎT DE CHASSEY, LYON (FR); LAURÈNE MEYNIEL-SCHICKLIN, LYON (FR); FRÉDÉRIC MIEGE, LYON (FR)

(21) Appl. No.: 18/039,985

(22) PCT Filed: Dec. 2, 2021
(86) PCT No.: PCT/EP2021/083891

§ 371 Date: Jun. 2, 2023

(30) Foreign Application Priority Data
Dec. 3, 2020 (EP) 20306485.2

(51) Int. Cl. C07D 403/14; A61P 31/16

(52) U.S. Cl. CPC C07D 403/14; A61P 31/16

(57) ABSTRACT

The present invention relates to pyrrole-type compounds of formula (I) and pharmaceutical compositions comprising the same. The invention further relates to such compounds and pharmaceutical compositions for use for treating a viral infection and/or cancer.

### FIELD OF THE INVENTION

The present invention relates to the field of medicine, in particular pyrrole-type compounds. The present invention further relates to pharmaceutical compositions comprising such pyrrole-type compounds and their uses for treating diseases such as viral infections and cancer.

### BACKGROUND OF THE INVENTION

Viruses are small infectious agents that replicates only inside living cells of other organisms. They can infect all types of life forms, from animals and plants to microorganisms, including bacteria and archaea. Among them, more than 400 species of virus are known to be responsible of diseases in humans, many of them leading to serious pathologies and eventually death. In particular, HIV was classified at the sixth leading cause of death worldwide in 2012 with 1.5 million of deaths per year (WHO, Fact sheet N°310, 2014). Seasonal influenza viruses are responsible of flu that affects approximately 20% of the world population and causes 250,000 to 500,000 deaths per year (WHO, Fact sheet N°211, 2014). Among other examples, Hepatitis B and C are responsible altogether for about 1.4 million of death each year and human Papillomaviruses are responsible of cervix cancer, the second most common women cancer worldwide, leading to 270,000 death in 2012 (WHO, Fact sheets, 2016).

Because viruses use vital metabolic pathways within host cells to replicate, they are difficult to eliminate without using drugs that cause toxic effects to host cells in general. The most effective medical approaches to viral diseases are vaccinations to provide immunity to infection, and antiviral drugs that selectively interfere with viral replication. Vaccines are very effective on stable viruses for a preventive use. However, vaccines are of limited use in treating a patient who has already been infected. They are also difficult to successfully deploy against rapidly mutating viruses, such as influenza (the vaccine for which is updated every year) and HIV. Antiviral drugs may be particularly useful in these cases.

Antiviral drugs are a class of medication used specifically for treating viral infections. Antiviral drugs do not destroy their target pathogens, instead they inhibit their development. Antiviral drugs may target any stage of the viral life cycle: attachment to a host cell, release of viral genes and possibly enzymes into the host cell, replication of viral components using host-cell machinery, assembly of viral components into complete viral particles, and release of viral particles to infect new host cells. The most common antiviral drugs are nucleoside analogues that block replication of viruses. Most antiviral drugs are used for specific viral infections, while broad-spectrum antiviral drugs are effective against a wide range of viruses.

Soon after the development of antiviral drugs, resistance appeared. Antiviral drug resistance can be defined as a decreased susceptibility to a drug through either a minimally effective, or completely ineffective, treatment response to prevent associated illnesses from a particular virus. Antiviral drug resistance remains a major obstacle to antiviral therapy as it has developed to almost all specific and effective antiviral drugs. For example, there are two main groups of antiviral drugs available for treatment and prophylaxis of influenza: M2 inhibitors (amantadine and rimantadine) and neuraminidase inhibitors (oseltamivir and zanamivir). Despite the effectiveness of these drugs in reducing influenza-related morbidity and mortality, the emergence of drug resistance poses a critical limitation on their application and have raised an urgent need for developing new anti-influenza drugs against resistant forms.

In this context, Denisova et al. have studied the potential activity against influenza A virus of anticancer compounds. More particularly, Denisova et al. have reported an antiviral effect of Obatoclax, which is known as a small-molecule antagonist of the Bcl-2 family of proteins, and gemcitabine (Denisova et al.: J. Biol. Chem., 2012, 287(42), 35324-32), making thereby such compounds as potent antiviral agents.

However, it still remains a strong need for the development of new antiviral drugs, and in particular broad-spectrum antiviral drugs. More particularly, the development of Obatoclax derivatives having an improved antiviral effect, remains a therapeutic approach to be investigated. The present invention seeks to meet these and other needs.

### SUMMARY OF THE INVENTION

As demonstrated by the comparative tests of the examples, the inventors have unexpectedly shown that derivatives of Obatoclax having a reduced indole core, i.e. a 4,5,6,7-tetrahydro-1H-indole, exhibited a higher antiviral effect compared to the Obatoclax referenced compound.

The present invention thus provides a new compound of formula (I):

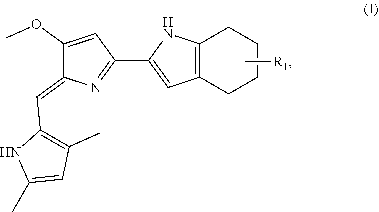

wherein R₁ represents a hydrogen, a (C₁-C₆)alkyl group, or a spiro-connected (C₃-C₆)cycloalkyl group, and the stereoisomers, and the pharmaceutical salts thereof.

In a preferred embodiment, R₁ represents a hydrogen, a methyl group, or a spiro-connected cyclopropyl group.

In a further preferred embodiment, the compound is of formula (I′):

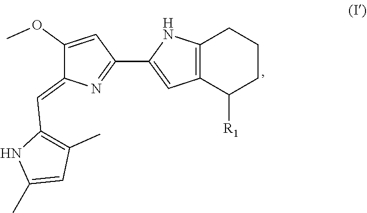

in which R₁ is such as disclosed herein.

In a more preferred embodiment, a compound of formula (I) is selected in the group consisting of:
(Z)-2-(2-((3,5-dimethyl-1H-pyrrol-2-yl)methylene)-3-methoxy-2H-pyrrol-5-yl)-4,5,6,7-tetrahydro-1H-indole (Compound 3);
(Z)-2-(2-((3,5-dimethyl-1H-pyrrol-2-yl)methylene)-3-methoxy-2H-pyrrol-5-yl)-4-methyl-4,5,6,7-tetrahydro-1H-indole (Compound 7);
(Z)-2-(2-((3,5-dimethyl-1H-pyrrol-2-yl)methylene)-3-methoxy-2H-pyrrol-5-yl)-5-methyl-4,5,6,7-tetrahydro-1H-indole (Compound 19); and
(Z)-2′-(2-((3,5-dimethyl-1H-pyrrol-2-yl)methylene)-3-methoxy-2H-pyrrol-5-yl)-1′,5′,6′,7′-tetrahydrospiro [cyclopropane-1,4′-indole] (Compound 23).

A further object of the invention is a compound of the invention for use as a medicine. Another object, is a pharmaceutical composition comprising a compound as defined herein, and a pharmaceutically acceptable excipient.

A particular aspect is a compound or a pharmaceutical composition according to the invention for use as antiviral agent or for treating a viral infection. In a particular embodiment, the viral infection is an infection by a virus selected from the group consisting of Heunggongvirae, Monodnaviria, Orthornavirae, Pararnavirae, and Shotokuvirae. In another particular embodiment, the viral infection is an infection by preferably selected from the group of Artverviricota, Cossaviricota, Duplornaviricota, Kitrinoviricota, Negarnaviricota, Peploviricota and Pisuviricota. In a further particular embodiment, the viral infection is an infection by a virus of a family selected from the group consisting of Coronaviridae, Flaviviridae, Hepadnaviridae, Herpesviridae, Orthomyxoviridae, Papillomaviridae, Paramyxoviridae, Picornaviridae, Polyomaviridae, Reoviridae, Retroviridae, Rhabdoviridae, and Togaviridae (e.g., Alphaviridae). In a preferred embodiment, the viral infection is an infection by a virus from Orthornavirae, more specifically Negarnaviricota, still more specifically Orthomyxoviridae. Preferably, the virus is selected from the group consisting of Influenza virus A, Influenza virus B, Influenza virus C, Isavirus, Thogotovirus and Quaranjavirus, preferably selected from the group consisting of Influenza virus A and Influenza virus B. Preferably, Influenza virus A is selected from the subtypes consisting of H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7.

A further particular aspect is a compound or a pharmaceutical composition according to the invention for use as an antitumor agent or for treating a cancer. Preferably, the cancer is selected from the group consisting of a breast cancer, a lung cancer, in particular NSCLC or SCLC, a melanoma, a colorectal cancer, an astrocytoma cancer, a liver cancer, leukemia, in particular acute myeloid leukemia or chronic lymphocytic leukemia, lymphoma, especially Hodgkin's lymphoma, a gastric cancer, a head and neck cancer, a cervical cancer, a pancreatic cancer, an ovarian cancer, myeloma and myelodysplastic syndromes such as myelofibrosis and mastocytosis.

### DETAILED DESCRIPTION OF THE INVENTION

### Definitions

According to the present invention, the terms below have the following meanings: The terms mentioned herein with prefixes such as for example C₁-C₃, C₁-C₆ or C₂-C₆ can also be used with lower numbers of carbon atoms such as C₁-C₂, C₁-C₅, or C₂-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms. If, for example, the term C₃-C₆ is used, it means that the corresponding hydrocarbon chain may comprise from 3 to 6 carbon atoms, especially 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl, an ethyl, a propyl, an isopropyl, or a tert-butyl, more preferably a methyl.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl, which can be spiro-connected to the rest of the molecule. In a preferred embodiment, the “cycloalkyl” is a cyclopropyl spiro-connected to the rest of the compound of formula (I).

The “stereoisomers” are isomeric compounds that have the same molecular formula and sequence of bonded atoms, but differ in the 3D-dimensional orientations of their atoms in space. The stereoisomers include enantiomers, diastereoisomers, Cis-trans and E-Z isomers, conformers, and anomers. In a preferred embodiment of the invention, the stereoisomers include diastereoisomers and enantiomers. The enantiomers compounds may be prepared from the racemate compound using any purification method known by a skilled person, such as LC/MS and chiral HPLC analysis methods and chiral SFC purification methods.

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic, tartaric and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, methanesulfonate, and tartrate, preferably tartrate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. In a preferred embodiment, the salt is selected from the group consisting of sodium and potassium salt.

As used herein, the terms “treatment”, “treat” or “treating” refer to any act intended to ameliorate the health status of patients such as therapy, prevention, prophylaxis and retardation of a disease, in particular a viral infection or a cancer, preferably a viral infection. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult, child, newborn and human at the prenatal stage. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a pharmaceutical composition having a therapeutic effect.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease, particularly a viral infection or a cancer. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

### Compounds

The present invention provides new compounds of therapeutic interest.

According to the invention, a compound has the following formula (I):

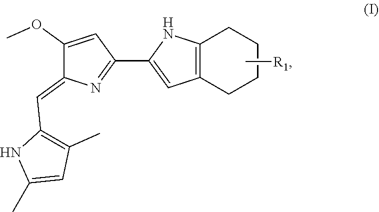

wherein R₁ represents a hydrogen, a (C₁-C₆)alkyl group, or a spiro-connected (C₃-C₆)cycloalkyl group, and the stereoisomers, and the pharmaceutical salts thereof.

In a particular embodiment, R₁ represents a hydrogen.

In a further particular embodiment, R₁ represents a (C₁-C₆)alkyl group, preferably a methyl, an ethyl, a propyl, or a butyl group, more preferably a methyl group.

In a further particular embodiment, R₁ represents a spiro-connected (C₃-C₆)cycloalkyl group, preferably a spiro-connected cyclopropyl group. A spiro-connected (C₃-C₆)cycloalkyl group may be represented as follows:

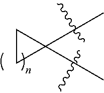

with n being an integer comprised between 1 and A spiro-connected cyclopropyl group may be represented as follows:

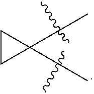 [SMILES: *C1(*)CC1]

Preferably, R₁ represents a hydrogen, a methyl group, or a spiro-connected cyclopropyl group.

In a preferred embodiment, a compound of the invention has the following formula (I′):

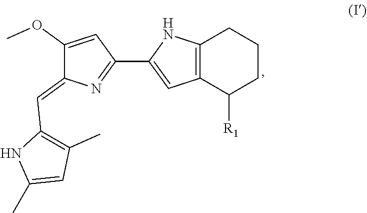

in which R₁ is such as above defined.

A more preferred compound of formula (I) is selected in the group consisting of:
(Z)-2-(2-((3,5-dimethyl-1H-pyrrol-2-yl)methylene)-3-methoxy-2H-pyrrol-5-yl)-4,5,6,7-tetrahydro-1H-indole (Compound 3);
(Z)-2-(2-((3,5-dimethyl-1H-pyrrol-2-yl)methylene)-3-methoxy-2H-pyrrol-5-yl)-4-methyl-4,5,6,7-tetrahydro-1H-indole (Compound 7);
(Z)-2-(2-((3,5-dimethyl-1H-pyrrol-2-yl)methylene)-3-methoxy-2H-pyrrol-5-yl)-5-methyl-4,5,6,7-tetrahydro-1H-indole (Compound 19); and
(Z)-T-(2-((3,5-dimethyl-1H-pyrrol-2-yl)methylene)-3-methoxy-2H-pyrrol-5-yl)-1′,5′,6′,7′-tetrahydrospiro[cyclopropane-1,4′-indole] (Compound 23).

A more preferred compound of formula (I) or (F) is selected in the group consisting of:
(Z)-2-(2-((3,5-dimethyl-1H-pyrrol-2-yl)methylene)-3-methoxy-2H-pyrrol-5-yl)-4,5,6,7-tetrahydro-1H-indole (Compound 3);
(Z)-2-(2-((3,5-dimethyl-1H-pyrrol-2-yl)methylene)-3-methoxy-2H-pyrrol-5-yl)-4-methyl-4,5,6,7-tetrahydro-1H-indole (Compound 7); and
(Z)-2′-(2-((3,5-dimethyl-1H-pyrrol-2-yl)methylene)-3-methoxy-2H-pyrrol-5-yl)-1′,5′,6′,7′-tetrahydrospiro[cyclopropane-1,4′-indole] (Compound 23).

The preferred compounds of the invention have the following formulae:

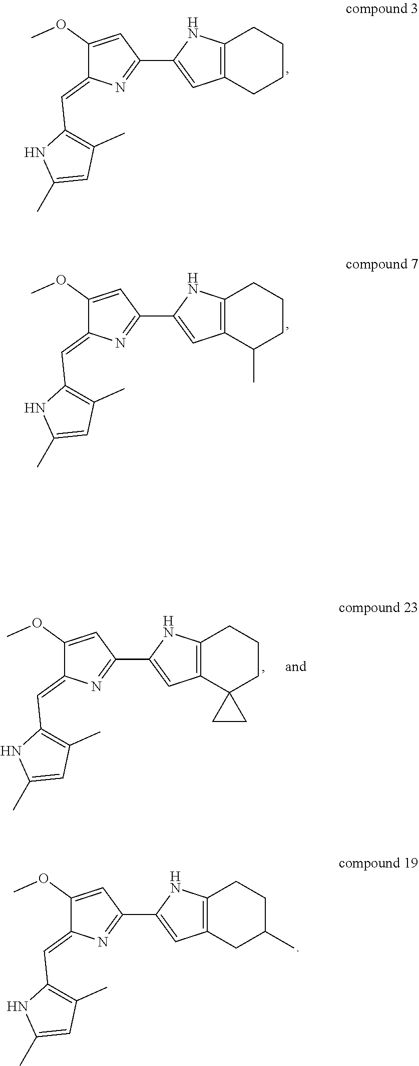

### Therapeutic Uses of Compounds

As illustrated by examples, the inventors have demonstrated the therapeutic interest of the new compounds of the invention.

Accordingly, the present invention relates to a pharmaceutical or veterinary composition comprising a new compound according to the invention. Preferably, the pharmaceutical composition further comprises a pharmaceutically or veterinary acceptable carrier or excipient. The present invention relates to a new compound according to the invention, for use as a drug or a medicine. The invention further relates to the use of a new compound according to the invention as a drug or a medicine. The invention further relates to a method for treating a disease in a subject, wherein a therapeutically effective amount of a new compound according to the invention, is administered to said subject in need thereof. The invention also relates to the use of a new compound according to the invention, for the manufacture of a medicine. The invention also relates to a pharmaceutical composition comprising a new compound according to the invention for use as a drug.

The present invention relates to a new compound according to the invention for use for treating a disease selected from the group consisting of a viral infection and a cancer. It further relates to the use of a new compound according to the invention, for the manufacture of a medicine for treating a disease selected from the group consisting of a viral infection and a cancer. It also relates to a pharmaceutical composition comprising a new compound according to the invention for use for treating a disease selected from the group consisting of a viral infection and a cancer. Finally, it relates to a method for treating a disease selected from the group consisting of a viral infection or a cancer in a subject in need thereof, wherein a therapeutically effective amount of a new compound according to the invention, is administered to said subject in need thereof.

In addition, the present invention relates to a method for treating a viral infection disease, in a subject, wherein a therapeutically effective amount of a compound according to the invention, is administered to said subject suffering of a viral infection disease. The present invention relates to the use of the compounds according to the invention as an antiviral agent. The invention also relates to the use of the compounds according to the invention, for the manufacture of a medicine for the treatment of a viral infection disease. The invention relates to a compound according to the invention for use in the treatment of a viral infection disease.

The present invention further relates to a method for treating a cancer in a subject, wherein a therapeutically effective amount of a compound according to the invention is administered to said subject suffering of a cancer. The present invention relates to the use of the compounds according to the invention as an antitumor agent. The invention also relates to the use of the compounds according to the invention, for the manufacture of a medicine for the treatment of a cancer. The invention relates to a compound according to the invention for use in the treatment of a cancer.

Antiviral Agents

The present invention relates to the use of a compound according to the invention as an antiviral agent. The present invention also relates to a compound of the present invention for use in the treatment of viral infections, the use of a compound of the present invention for the manufacture of a medicine for the treatment of viral infections, and to a method for treating a viral infection in a subject, comprising administering a therapeutically effective amount of a compound according to the invention to the subject.

The present invention also relates to the use of a compound of the present invention as a research tool, especially for studying viral infections. It further relates to a method for blocking viral infection in a cell, a tissue or a subject.

The viral agent can be a DNA virus or a RNA virus. The viral agent may belong to a virus from the group consisting of Heunggongvirae, Monodnaviria, Orthornavirae, Pararnavirae, and Shotokuvirae. More specifically, the viral agent may belong to a virus selected from the group consisting of Artverviricota, Cossaviricota, Duplornaviricota, Kitrinoviricota, Negarnaviricota, Peploviricota and Pisuviricota. The viral agent can be selected from the group consisting of Coronaviridae, Flaviviridae, Hepadnaviridae, Herpesviridae, Orthomyxoviridae, Papillomaviridae, Paramyxoviridae, Picornaviridae, Polyomaviridae, Reoviridae, Retroviridae, Rhabdoviridae, and Togaviridae (e.g., Alphaviridae), Tobamoviruses. A particular embodiment of the invention is a compound or a pharmaceutical composition for use for treating a viral infection by a virus selected from the group consisting of Heunggongvirae, Monodnaviria, Orthornavirae, Pararnavirae, and Shotokuvirae; or selected from the group consisting of Artverviricota, Cossaviricota, Duplornaviricota, Kitrinoviricota, Negarnaviricota, Peploviricota and Pisuviricota; or selected from the group consisting of Coronaviridae, Flaviviridae, Hepadnaviridae, Herpesviridae, Orthomyxoviridae, Papillomaviridae, Paramyxoviridae, Picornaviridae, Polyomaviridae, Reoviridae, Retroviridae, Rhabdoviridae, and Togaviridae (e.g., Alphaviridae). Preferably, the virus is Orthomyxoviridae.

In one embodiment, the Coronaviridae can be an Orthocoronavirinae and can belong to the genus alphacoronavirus, betacoronavirus, deltacoronavirus and gammacoronavirus.

In one embodiment, the Togaviridae and especially Alphaviridae is selected from the group consisting of Barmah Forest virus, Middelburg virus, Ndumu virus, Bebaru virus, Chikungunya virus, Mayaro virus, O'nyong'nyong virus, Ross River virus, Semliki Forest virus, Sindbis virus, Una virus, Eastern equine encephalitis virus, Tonate virus, Venezuelan equine encephalitis virus, Cabassou virus, Everglades virus, Mosso das Pedras virus, Mucambo virus, Parmana virus, Pixuna virus, Rio Negro virus, Trocara virus, Aura virus, Babanki virus, Kyzylagach virus, Ockelbo virus, Whataroa virus, Sleeping disease virus, Samon pancreatic disease virus, Southern elephant seal virus, and Western equine encephalitis virus; preferably selected from the group consisting of Barmah Forest virus, Chikungunya virus, Mayaro virus, O'nyong'nyong virus, Ross River virus, Semliki Forest virus, Sindbis virus, Una virus, Eastern equine encephalitis virus, Tonate virus, Venezuelan equine encephalitis virus and Western equine encephalitis virus.

In one embodiment, the Flaviviridae is selected from the group consisting of dengue virus, Hepatitis C virus, Japanese encephalitis virus, West Nile virus, yellow fever virus, Zika virus, Tick-borne encephalitis virus, Kyasanur forest disease virus, Murray Valley encephalitis virus, and Saint Louis encephalitis virus.

In one embodiment, the Hepadnaviridae is selected from the group consisting of Hepatitis B virus.

In one embodiment, the Herpesviridae is selected from the group consisting of Herpes Simplex virus 1 (HSV-1), Herpes Simplex virus 2 (HSV-2), Varicella zoster virus (VZV), Epstein-Barr virus (EBV), Cytomegalovirus (CMV), Roseolovirus (HHV-6A and 6B), HHV-7 and Kaposi's sarcoma-associated herpesvirus (KSHV).

In one embodiment, the Orthomyxoviridae is selected from the group consisting of Influenza virus A, Influenza virus B, Influenza virus C, Isavirus, Thogotovirus and Quaranjavirus, preferably selected from the group consisting of Influenza virus A and Influenza virus B. In one embodiment, the Influenza virus A is selected from the subtypes consisting of H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7.

In one embodiment, the Polyomavirus is selected from the group consisting of Simian virus Merkel cell polyomavirus, Trichodysplasia spinulosa polyomavirus, BK polyomavirus, JC polyomavirus and Human polyomavirus 7.

In one embodiment, the Paramyxoviridae is selected from the group consisting of Rubulavirus, Morbillivirus, Pneumovirus, Metapneumovirus, Avulavirus, Ferlavirus, Henipavirus, and Respirovirus. In a particular embodiment, the Paramyxoviridae is the mumps virus, measles virus, human parainfluenza viruses (HPIV), especially HPIV-1, HPIV-2, HPIV-3 or HPIV-4, respiratory syncytial virus (RSV), in particular Human respiratory syncytial virus (HRSV), canine distemper virus, phocine distemper virus, cetacean morbillivirus, Newcastle disease virus, rinderpest virus, Hendra birus and Nipah virus.

In one embodiment, the Picornaviridae is selected from the group consisting of Aphthovirus, Aquamavirus, Avihepatovirus, Cardiovirus, Cosavirus, Dicipivirus, Enterovirus, Erbovirus, Hepatovirus, Kobuvirus, Megrivirus, Parechovirus, Piscevirus, Rhinovirus, Salivirus, Sapelovirus, Senecavirus, Techovirus, and Tremovirus. In a particular embodiment, the Picornaviridae is a Rhinovirus, for instance a Rhinovirus A, Rhinovirus B or Rhinovirus C.

In one embodiment, the Retroviridae is selected from the group consisting of Alpharetrovirus; especially Avian leukosis virus and Rous sarcoma virus; Betaretrovirus, especially Mouse mammary tumour virus; Gammaretrovirus, especially Murine leukemia virus and Feline leukemia virus; Deltaretrovirus, especially Bovine leukemia virus and Human T-lymphotropic virus; Epsilonretrovirus, especially Walleye dermal sarcoma virus; Lentivirus, especially Human immunodeficiency virus 1 and Simian, Feline immunodeficiency viruses; Spumavirus, especially Simian foamy virus.

In one embodiment, the Rhabdoviridae is selected from the group consisting of vesiculovirus, especially vesicular stomatitis virus, lyssavirus, rabies virus, Ephemerovirus, novirhabdovirus, cytorhabdovirus and nucleorhabdovirus.

In one preferred embodiment, the viral agent according to the invention is selected from the group consisting in Herpesviridae such as Varicella zoster virus (VZV), Epstein-Barr (EB) virus, Herpes simplex virus of type 1 (HSV-1), Kaposis sarcoma herpesvirus (KSHV), murine γ-HV68 virus (γ-MHV68), or human cytomegalovirus (HCMV); Hepadnaviridae such as Hepatitis virus B (HBV); Papovaviridae such as Human papillomavirus type 16 (HPV16); Parvoviridae such as Human parvovirus B19; Polyomaviridae such as Simian virus 40; Retroviridae such has Human immunodeficiency virus 1 (HIV-1), or Simian immunodeficiency virus type 1 (SIV 1); Orthomyxoviridae such as Influenza A virus; Flaviviridae such as Dengue virus, or Hepatitis C virus; Picornaviridae such as Poliovirus, Coxsakievirus B3 (CVB3), or Coxsakievirus B4 (CVB4); Reoviridae such as Rotavirus; Alphaviridae such as Sindbis virus; Rhabdoviridae such as vesicular stomatitis virus. More preferably, the viral agent according to the invention is an influenza virus. Still preferably, the viral agent according to the invention is an influenza virus A or B, even more preferably an influenza virus A.

In another preferred embodiment, the viral agent according to the invention presents an antiviral resistance to classic antiviral drugs. The terms “antiviral resistance”, “antiviral agent resistance” or “antiviral drug resistance”, as used herein, are equivalent and refer to the ability of viruses to resist the effects of an antiviral agent previously used to treat them. Antiviral resistance can be defined by a decreased susceptibility to a drug through either a minimally effective, or completely ineffective, treatment response to prevent associated illnesses from a particular virus.

In one embodiment, the compound of the invention can be used in combination with another antiviral drug, for instance and non-exhaustively, an agent selected from the group consisting of neuraminidase inhibitors, M2 inhibitors, RNA polymerase inhibitors, interferons (immune system modulators interferon alpha-2a and PEGylated interferon alpha-2a (Pegasys) and interferon alpha-2b (ViraferonPeg ou Introna)), antiviral vaccine, antigenic polypeptides or neutralizing antibodies directed to a viral antigenic polypeptide.

Cancers

The compounds of the present invention are able to kill tumor cells. Accordingly, the present invention relates to the use of a compound of the present invention as an antitumor agent. The present invention also relates to a compound of the present invention for use for treating a cancer, the use of a compound of the present invention for the manufacture of a medicine for treating a cancer, and a method for treating a cancer in a subject, comprising administering an effective amount of a compound of the present invention to the subject.

In one aspect, the cancer can be a solid tumor or a hematopoietic cancer. For instance, the cancer can be selected from the group consisting of bone cancer, gastrointestinal cancer, liver cancer, pancreatic cancer, gastric cancer, colorectal cancer, an astrocytoma cancer, esophageal cancer, oro-pharyngeal cancer, laryngeal cancer, salivary gland carcinoma, thyroid cancer, lung cancer, cancer of the head or neck, skin cancer, squamous cell cancer, melanoma, uterine cancer, cervical cancer, endometrial carcinoma, vulvar cancer, ovarian cancer, breast cancer, prostate cancer, cancer of the endocrine system, sarcoma of soft tissue, bladder cancer, kidney cancer, glioblastoma and various types of cancers of the central nervous system, lymphoma and leukemia. Preferably, the cancer is selected from the group consisting of a breast cancer, a lung cancer, in particular SCLC (small cell lung cancer) and NSCLC (non-small cell lung cancer), a melanoma, a colorectal cancer, an astrocytoma cancer, a liver cancer, leukemia, in particular acute myeloid leukemia or chronic lymphocytic leukemia, B-cell chronic lymphocytic leukemia, lymphoma, such as follicular lymphoma, non-Hodgkin's lymphoma, Hodgkin's lymphoma, recurrent adult diffuse large cell lymphoma, a gastric cancer, a head and neck cancer, a cervical cancer, a pancreatic cancer, an ovarian cancer, myeloma, especially multiple myeloma and myelodysplastic syndromes such as myelofibrosis and mastocytosis.

In a particular aspect, the compound of the present invention can be combined with radiotherapy, immunotherapy, hormonotherapy, or chemotherapy, all well-known by the person skilled in the field.

Pharmaceutical Composition

The present invention also relates to a pharmaceutical composition comprising a compound of the present invention. The composition further comprises at least one pharmaceutically acceptable carrier or excipient.

In a particular embodiment, the pharmaceutical composition according to the invention further comprises at least another active ingredient, preferably selected from the group consisting of an antiviral agent and an anti-cancerous agent. Preferably, the other active ingredient is an antiviral agent. More preferably, the other active ingredient is an antiviral agent against an influenza virus, preferably an influenza A virus.

In a particular embodiment, the pharmaceutical composition according to the invention further comprises an antiviral agent, for instance and non-exhaustively, an agent selected from the group consisting of neuraminidase inhibitors, M2 inhibitors, RNA polymerase inhibitors, interferons (immune system modulators interferon alpha-2a and PEGylated interferon alpha-2a (Pegasys) and interferon alpha-2b (ViraferonPeg ou Introna)), antiviral vaccine, antigenic polypeptides or neutralizing antibodies directed to a viral antigenic polypeptide.

The invention also concerns the pharmaceutical composition of the invention for use in the treatment of a disease. The invention also relates to the use of a pharmaceutical composition according to the invention for the manufacture of a medicine for treating a disease in a subject. The invention further relates to a method for treating a disease in a subject, wherein a therapeutically effective amount of a pharmaceutical composition according to the invention is administered to said subject suffering from said disease.

The subject according to the invention is an animal, preferably a mammal, even more preferably a human. However, the term “subject” can also refer to non-human animals, in particular mammals such as dogs, cats, horses, cows, pigs, sheep, donkeys, rabbits, ferrets, gerbils, hamsters, chinchillas, rats, mice, guinea pigs and non-human primates, among others, that are in need of treatment.

The human subject according to the invention may be a human at the prenatal stage, a newborn, a child, an infant, an adolescent or an adult.

In a preferred embodiment, the subject has been diagnosed with a disease. Preferably, the subject has been diagnosed with a disease selected from the group consisting in viral infections, and cancers.

The compound according to the invention or the pharmaceutical composition according to the invention may be administered by any conventional route of administration. In particular, the compound or the pharmaceutical composition of the invention can be administered by a topical, enteral, oral, parenteral, intranasal, intravenous, intra-arterial, intramuscular, intratumoral, subcutaneous or intraocular administration and the like.

In particular, the compound according to the invention or the pharmaceutical composition according to the invention can be formulated for a topical, enteral, oral, parenteral, intranasal, intravenous, intra-arterial, intramuscular, intratumoral, subcutaneous or intraocular administration and the like.

Preferably, the compound according to the invention or the pharmaceutical composition according to the invention is administered by enteral or parenteral route of administration. When administered parenterally, the compound according to the invention or the pharmaceutical composition according to the invention is preferably administered by intravenous route of administration. When administered enterally, the compound according to the invention or the pharmaceutical composition according to the invention is preferably administered by oral route of administration.

The pharmaceutical composition comprising the molecule is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatin, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

Preferably, the treatment with the compound according to the invention or the pharmaceutical composition according to the invention start no longer than a month, preferably no longer than a week, after the diagnosis of the disease. In a most preferred embodiment, the treatment starts the day of the diagnosis.

The compound according to the invention or the pharmaceutical composition according to the invention may be administered as a single dose or in multiple doses.

Preferably, the treatment is administered regularly, preferably between every day and every month, more preferably between every day and every two weeks, more preferably between every day and every week, even more preferably the treatment is administered every day. In a particular embodiment, the treatment is administered several times a day, preferably 2 or 3 times a day, even more preferably 3 times a day.

The duration of treatment with the compound according to the invention or the pharmaceutical composition according to the invention is preferably comprised between 1 day and 20 weeks, more preferably between 1 day and 10 weeks, still more preferably between 1 day and 4 weeks, even more preferably between 1 day and 2 weeks. In a particular embodiment, the duration of the treatment is of about 1 week. Alternatively, the treatment may last as long as the disease persists.

The amount of compound according to the invention or of pharmaceutical composition according to the invention to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a preferred embodiment, the total compound dose for each administration of the compound according to the invention or of the pharmaceutical composition according to the invention is comprised between 0.00001 and 1 g, preferably between 0.01 and 10 mg.

The form of the pharmaceutical compositions, the route of administration and the dose of administration of the compound according to the invention, or the pharmaceutical composition according to the invention can be adjusted by the man skilled in the art according to the type and severity of the disease, and to the patient, in particular its age, weight, sex, and general physical condition.

Kit and Use of a Kit

The present invention also relates to the combined use of a compound of the present invention with at least another active ingredient, preferably selected from the group consisting of an antiviral agent, an anti-cancerous agent, an anti-apoptotic agent, an anti-autophagy agent, and an autophagy inducing agent, for the treatment of a disease selected from the group consisting of cancer and infectious viral diseases.

The present invention also relates to a product comprising a compound of the present invention, and another active ingredient, as a combined preparation for simultaneous, separate or sequential use, in particular for use for the treatment of a disease selected from the group consisting of cancer and viral infectious diseases. Preferably, the other active ingredient is selected from the group consisting of an antiviral agent, an anti-cancerous agent, an anti-apoptotic agent, an anti-autophagy agent, an autophagy inducing agent, or a molecule aimed to treat cancer, infectious viral diseases. Preferably, the other active ingredient is an antiviral.

Further aspects and advantages of the present invention will be described in the following examples, which should be regarded as illustrative and not limiting.

### EXAMPLES

### Example A—Chemistry

### (Z)-2-(2-(2(3,5-dimethyl-1H-pyrrol-2-yl)methylene)-3-methoxy-2H-pyrrol-5-yl)-4,5,6,7-tetrahydro-1H-indole (Compound 3)

### Synthesis of 4,5,6,7-tetrahydro-1H-indole (Compound 1)

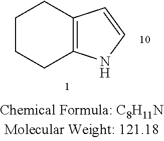

in dry THF (15 mL) at RT, was added LiAlH₄ (2M solution in THF, 3.26 mL, 6.51 mmol, 2.2 equiv) dropwise. The mixture was stirred at reflux for 4 h. After cooling down to 0° C., water (250 μL), 15% aq NaOH (250 μL) and water (750 μL) were successively added and the suspension was stirred at RT for 0.5 h. MTBE was added and the suspension was filtered over Celite (MTBE rinses) and the filtrate was concentrated under reduced pressure. The residue was purified by Flash Chromatography (cHex/EA=100/0 to 80/20) to afford 312 mg (87%) of compound 1 as a light pink oil. ¹HNMR (CDCl₃, 500 MHz): δ 7.72 (br s, 1H), 6.66 (app t, J=2.7 Hz, 1H), 6.03 (app t, J=2.7 Hz, 1H), 2.62 (m, 2H), 2.55 (m, 2H), 1.90-1.83 (m, 2H), 1.83-1.76 (m, 2H).

### Synthesis of 3-methoxy-5-(4,5,6,7-tetrahydro-1H-indol-2-yl)-1H-pyrrole-2-carbaldehyde (Compound 2)

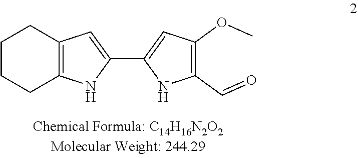 [SMILES: COc1cc(-c2cc3c([nH]2)CCCC3)[nH]c1C=O]

To a solution of compound 1 (125 mg, 1.03 mmol) in hexane (3 mL) at RT, were added [Ir(COD)OMe]₂ (14 mg, 0.021 mmol, 0.02 equiv), 4,4′-Di-tert-butyl-2,2′-dipyridyl (11 mg, 0.041 mmol, 0.07 equiv) and B₂pin₂ (154 mg, 0.619 mmol, 0.6 equiv). The reaction mixture was stirred at RT for 1.5 h. The reaction mixture was concentrated under reduced pressure until ca. 1 mL hexane left. Degassed dioxane/H₂O (9/1, 5 mL), Pd(PPh₃)₄ (89 mg, 0.077 mmol, 0.07 equiv), Na₂CO₃ (328 mg, 3.09 mmol, 3 equiv) and (E)-1-(5-bromo-3-methoxy-2H-pyrrol-2-ylidene)-N,N-dimethylmethanamine (200 mg, 0.774 mmol, 0.75 equiv) were added and the reaction mixture was stirred at 80° C. for 5 h. After cooling down to RT, the reaction mixture was poured on water and 1N HCl was added until pH=7. The aqueous phase was extracted with DCM. The combined organic extracts were dried (Na₂SO₄), filtered and concentrated under reduced pressure. The residue was triturated in a biphasic mixture of EtOAc (4 mL) and water (4 mL) for 0.5 h. The resulting slurry was filtered over fritted glass and the filter cake was washed with water (3*2 mL) and EtOAc (3*2 mL). The yellow cake was dried under vacuum for 1 h to afford 140 mg (74%) of compound 2 as a yellow solid. ¹HNMR (DMSO-d₆, 500 MHz): δ 11.19 (br s, 1H), 10.76 (br s, 1H), 9.25 (s, 1H), 6.47 (s, 1H), 6.18 (s, 1H), 3.83 (s, 3H), 2.56 (m, 2H), 2.42 (m, 2H), 1.82-1.60 (m, 4H).

### Synthesis of (Z)-2-(2-((3,5-dimethyl-1H-pyrrol-2-yl)methylene)-3-methoxy-2H-pyrrol-5-yl)-4,5,6,7-tetrahydro-1H-indole (Compound 3)

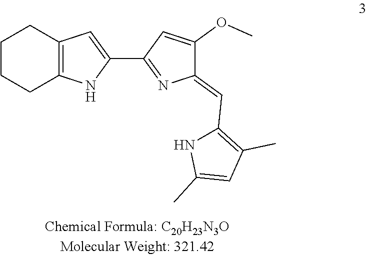 [SMILES: COC1=CC(c2cc3c([nH]2)CCCC3)=N/C1=C\c1[nH]c(C)cc1C]

To a suspension of compound 2 (76 mg, 0.31 mmol) in MeOH (1.25 mL) at RT, were added 2,4-dimethylpyrrole (36 mg, 0.37 mmol, 1.2 equiv) and HCl (0.5N solution in MeOH, 0.81 mL, 0.40 mmol, 1.3 equiv). The reaction mixture was stirred at RT for 64 h. The reaction mixture was filtered over fritted glass and the grey filter cake was washed with MeOH (3*2 mL). The filter cake was collected and suspended in MeOH (1 mL). Saturated NH₄OH solution in water was added (0.1 mL) and the resulting suspension was stirred at RT for 15 min. It was filtered over fritted glass and the filter cake was washed with MeOH (2*1 mL), collected and dried under vacuum to afford 46 mg (46%) of compound 3 as a red solid. ¹HNMR (CDCl₃, 500 MHz): δ 6.83 (s, 1H), 6.37 (s, 1H), 6.03 (s, 1H), 5.65 (s, 1H), 3.98 (s, 3H), 2.45 (br s, 2H), 2.19 (br s, 5H), 1.76 (br s, 3H), 1.65 (br s, 4H). MS (ESI⁺): [M+H]⁺ 322.

### (Z)-2-(2-(2-(3,5-dimethyl-1H-pyrrol-2-yl)methylene)-3-methoxy-2H-pyrrol-5-yl)-4-methyl-4,5,6,7-tetrahydro-1H-indole (compound 7)

### Synthesis of 4-methylene-1-tosyl-4,5,6,7-tetrahydro-1H-indole (Compound 4)

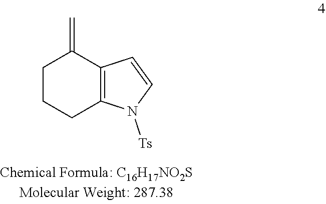 [SMILES: C=C1CCCc2c1ccn2S(=O)(=O)c1ccc(C)cc1]

To suspension of methyltriphenylphosphonium bromide (925 mg, 2.59 mmol, 1.5 equiv) in THF (9 mL) at RT, was added t-BuOK (291 mg, 2.59 mmol, 1.5 equiv). The resulting yellow suspension was stirred at RT for 15 min before 1-tosyl-1,5,6,7-tetrahydro-4H-indol-4-one (500 mg, 1.73 mmol) was added. After stirring for 30 min at RT, the reaction mixture was concentrated under reduced pressure. The residue was purified by Flash Chromatography (cHex/EA=100/0 to 80/20) to afford 466 mg (94%) of compound 4 as a colorless oil. ¹HNMR (CDCl₃, 500 MHz): δ 7.71 (m, 2H), 7.32 (m, 2H), 7.22 (d, J=3.4 Hz, 1H), 6.43 (d, J=3.5 Hz, 1H), 5.06 (s, 1H), 4.78 (s, 1H), 2.80 (t, J=6.2 Hz, 1H, 2H), 2.44 (s, 3H), 2.35 (m, 2H), 1.83 (m, 2H).

### Synthesis of 4-methyl-4,5,6,7-tetrahydro-1H-indole (Compound 5)

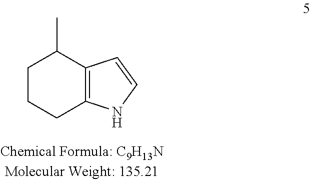

A suspension of compound 4 (390 mg, 1.36 mmol) and Pd/C (7.2 mg, mmol, 0.05 equiv) in THF/MeOH (1/1, 7 mL) was stirred under an atmosphere of H₂ for 1 h. The reaction mixture was filtered over Celite (THF rinses) and the filtrate was concentrated under reduced pressure. The residue was dissolved in THF/MeOH (1/1, 14 mL) and Mg (331 mg, 13.6 mmol, 10 equiv) and NH₄Cl (146 mg, 2.72 mmol, 2 equiv) were added. The reaction mixture was stirred at RT for 16 h before being partitioned between aq. sat. NH₄Cl and EtOAc. The layers were separated and the aqueous phase was extracted with EtOAc. The combined organic extracts were washed (brine), dried (Na₂SO₄), filtered and concentrated under reduced pressure. The residue was purified by Flash Chromatography (cHex/EA=100/0 to 80/20) to afford 126 mg (69%) of 5 as a light red oil. ¹HNMR (CDCl₃, 500 MHz): δ 7.61 (br s, 1H), 6.56 (app t, J=2.7 Hz, 1H), 5.99 (app t, J=2.7 Hz, 1H), 2.65 (m, 1H), 2.54-2.44 (m, 2H), 1.91-1.79 (m, 2H), 1.70-1.58 (m, 1H), 1.28-1.16 (m, 1H), 1.12 (d, J=6.9 Hz, 3H).

### Synthesis of 3-methoxy-5-(4-methyl-4,5,6,7-tetrahydro-1H-indol-2-yl)-1H-pyrrole-2-carbaldehyde (Compound 6)

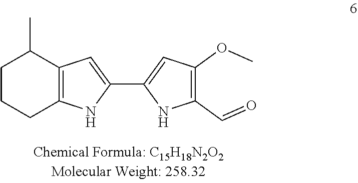 [SMILES: COc1cc(-c2cc3c([nH]2)CCCC3C)[nH]c1C=O]

(2.5 mL) at RT, were added [Ir(COD)OMe]2 (11 mg, 0.016 mmol, equiv), 4,4′-Di-tert-butyl-2,2′-dipyridyl (8.6 mg, 0.032 mmol, 0.04 equiv) and B₂pin₂ (123 mg, 0.484 mmol, 0.6 equiv). The reaction mixture was stirred at RT for 2 h. The reaction mixture was concentrated under reduced pressure until ca. 1 mL hexane left. Degassed dioxane/H₂O (9/1, 4.3 mL), Pd(PPh₃)₄ (70 mg, 0.060 mmol, 0.07 equiv), Na₂CO₃ (256 mg, 2.42 mmol, 3 equiv) and (E)-1-(5-bromo-3-methoxy-2H-pyrrol-2-ylidene)-N,N-dimethylmethanamine (157 mg, 0.604 mmol, 0.75 equiv) were added and the reaction mixture was stirred at 80° C. for 5 h. After cooling down to RT, EtOAc (6 mL), water (6 mL) and 2N HCl (1.2 mL to reach pH=7) were added and the suspension was stirred for 0.5 h. The resulting slurry was filtered over fritted glass and the filter cake was washed with water (3*2 mL) and EtOAc (3*2 mL). The yellow cake was dried under vacuum for 1 h to afford 80 mg (51%) of 6 as an orange solid. ¹HNMR (DMSO-d₆, 500 MHz): δ 11.17 (br s, 1H), 10.75 (br s, 1H), 9.25 (s, 1H), 6.58 (s, 1H), 6.18 (d, J=2.5 Hz, 1H), 3.83 (s, 3H), 2.67-2.57 (m, 1H), 2.57-2.50 (m, 2H), 1.94-1.80 (m, 2H), 1.70-1.58 (m, 1H), 1.28-1.16 (m, 1H), 1.13 (d, J=6.9 Hz, 3H).

### Synthesis of (Z)-2-(2-((3,5-dimethyl-1H-pyrrol-2-yl)methylene)-3-methoxy-2H-pyrrol-5-yl)-4-methyl-4,5,6,7-tetrahydro-1H-indole (Compound 7)

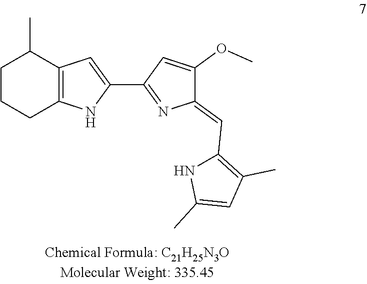 [SMILES: COC1=CC(c2cc3c([nH]2)CCCC3C)=N/C1=C\c1[nH]c(C)cc1C]

To a suspension of compound 6 (80 mg, 0.31 mmol) in MeOH (1.25 mL) at RT, were added 2,4-dimethylpyrrole (59 mg, 0.62 mmol, 2.0 equiv) and HCl (1.25N solution in MeOH, 0.37 mL, 0.46 mmol, 1.5 equiv). The reaction mixture was stirred at RT for 24 h. The reaction mixture was filtered over fritted glass and the grey filter cake was washed with MeOH (3*2 mL). The filter cake was collected and suspended in MeOH (1 mL). Saturated NH₄OH solution in water was added (0.5 mL) and the resulting suspension was stirred at RT for 15 min. It was filtered over fritted glass and the filter cake was washed with MeOH (2*1 mL), collected and dried under vacuum to afford 33 mg (32%) of compound 7 as a red solid. ¹HNMR (CDCl₃, 500 MHz): δ 6.82 (s, 1H), 6.45 (s, 1H), 6.04 (s, 1H), 5.66 (s, 1H), 3.97 (s, 3H), 2.63 (br s, 1H), 2.35-2.10 (m, 5H), 1.90-1.45 (m, 6H), 1.30-1.00 (m, 4H). MS (ESI⁺): [M+H]⁺ 336.

### (Z)-2-(2-(2-(3,5-dimethyl-1H-pyrrol-2-yl)methylene)-3-methoxy-2H-pyrrol-5-yl)-5-methyl-4,5,6,7-tetrahydro-1H-indole (Compound 19)

### Synthesis of 5-methyl-4,5,6,7-tetrahydro-1H-indole (Compound 17)

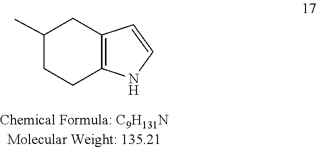

To a solution of tert-butyl 4-oxo-4,5,6,7-tetrahydro-1H-indole-1-carboxylate (500 mg, 2.13 mmol) in THF (8.5 mL) at −78° C., was added dropwise LiHMDS (1.0M solution in THF, 2.23 mL, 2.23 mmol, 1.05 equiv). The yellow solution was stirred −78° C. for 1 h and Mel (0.197 mL, 3.19 mmol, 1.5 equiv) was added dropwise. The mixture was stirred at −78° C. for 0.25 h and then allowed to warm-up to RT and stirred at RT for 0.5 h. Sat aq. NH₄Cl and DCM were added. The layers were separated and the aqueous phase was extracted with DCM. The combined organic extracts were dried (Na₂SO₄), filtered and concentrated under reduced pressure. The residue was dissolved in DCM (4.3 mL) at RT and TFA (3.26 mL, 42.6 mmol, 20 equiv) was added. The mixture was stirred at RT for 2 h. K₂CO₃(s) was added portion wise until neutralization and the resulting suspension was filtered (DCM rinses). The filtrate was concentrated under reduced pressure. The residue was solubilized in dry THF (10.5 mL) at RT, and LiAlH₄ (1M solution in THF, 4.69 mL, 4.69 mmol, 2.2 equiv) was added dropwise. The mixture was stirred at reflux for 4 h. After cooling down to 0° C., water (200 μL), 15% aq NaOH (200 μL) and water (600 μL) were successively added and the suspension was stirred at RT for 0.5 h. MTBE was added and the suspension was filtered over Celite (MTBE rinses) and the filtrate was concentrated under reduced pressure. The residue was purified by Flash Chromatography (cHex/EA=100/0 to 80/20) to afford 207 mg (87%) of 17 as a light yellow solid. ¹HNMR (CDCl₃, 500 MHz): δ 7.62 (br s, 1H), 6.55 (app t, J=2.7 Hz, 1H), 5.89 (app t, J=2.7 Hz, 1H), 2.60-2.50 (m, 3H), 2.10-2.02 (m, 1H), 1.83-1.70 (m, 2H), 1.44-1.32 (m, 1H), 0.98 (d, J=6.6 Hz, 3H).

### Synthesis of 3-methoxy-5-(5-methyl-4,5,6,7-tetrahydro-1H-indol-2-yl)-1H-pyrrole-2-carbaldehyde (Compound 18)

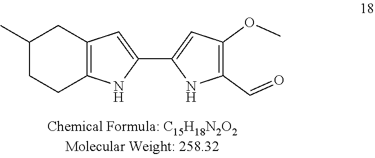

To a solution of compound 17 (109 mg, 0.806 mmol) in hexane (2.5 mL) at RT, were added [Ir(COD)OMe]2 (11 mg, 0.016 mmol, equiv), 4,4′-Di-tert-butyl-2,2′-dipyridyl (8.7 mg, 0.032 mmol, 0.07 equiv) and B₂pin₂ (123 mg, 0.484 mmol, 0.6 equiv). The reaction mixture was stirred at RT for 2 h. The reaction mixture was concentrated under reduced pressure until ca. 1 mL hexane left. Degassed dioxane/H₂O (9/1, 4.3 mL), Pd(PPh₃)₄ (70 mg, 0.060 mmol, 0.07 equiv), Na₂CO₃ (256 mg, 2.42 mmol, 3 equiv) and (E)-1-(5-bromo-3-methoxy-2H-pyrrol-2-ylidene)-N,N-dimethylmethanamine (157 mg, 0.604 mmol, 0.75 equiv) were added and the reaction mixture was stirred at 80° C. for 5 h. After cooling down to RT, EtOAc (6 mL), water (6 mL) and 2N HCl (1.2 mL to reach pH=7) were added and the suspension was stirred for 0.5 h. The resulting slurry was filtered over fritted glass and the filter cake was washed with water (3*2 mL) and EtOAc (3*2 mL). The yellow cake was dried under vacuum for 1 h to afford 142 mg (91%) of compound 18 as a yellow solid. ¹HNMR (DMSO-d₆, 500 MHz): δ 11.18 (br s, 1H), 10.76 (br s, 1H), 9.25 (s, 1H), 6.45 (s, 1H), 6.17 (d, J=2.5 Hz, 1H), 3.83 (s, 3H), 2.62-2.55 (m, 2H), 2.55-2.48 (m, 1H), 2.03 (dd, J=15.3&9.9 Hz, 1H), 1.87-1.70 (m, 2H), 1.45-1.30 (m, 1H), 1.02 (d, J=6.6 Hz, 3H).

### Synthesis of (Z)-2-(2-((3,5-dimethyl-1H-pyrrol-2-yl)methylene)-3-methoxy-2H-pyrrol-5-yl)-5-methyl-4,5,6,7-tetrahydro-1H-indole (Compound 19)

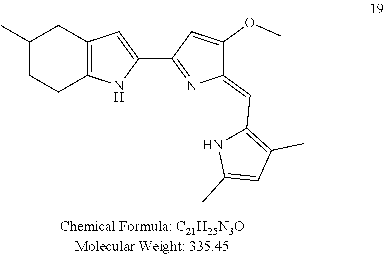

To a suspension of compound 18 (84 mg, 0.32 mmol) in MeOH (1.25 mL) at RT, were added 2,4-dimethylpyrrole (62 mg, 0.65 mmol, 2.0 equiv) and HCl (1.25N solution in MeOH, 0.39 mL, 0.49 mmol, 1.5 equiv). The reaction mixture was stirred at RT for 24 h. The reaction mixture was filtered over fritted glass and the grey filter cake was washed with MeOH (3*2 mL). The filter cake was collected and suspended in MeOH (5 mL). Saturated NH₄OH solution in water was added (0.5 mL) and the resulting suspension was stirred at RT for 15 min. It was filtered over fritted glass and the filter cake was washed with MeOH (2*1 mL), collected and dried under vacuum to afford 40 mg (36%) of compound 19 as a red solid. ¹HNMR (CDCl₃, 500 MHz): δ 6.82 (s, 1H), 6.35 (s, 1H), 6.02 (s, 1H), 5.65 (s, 1H), 3.97 (s, 3H), 2.53 (app d, J=12.6 Hz, 1H), 2.40-2.10 (br m, 2H), 2.05 (m, 1H), 2.18 (s, 3H), 1.90-1.60 (m, 5H), 1.35-1.20 (m, 1H), 1.10-0.85 (d, J=6.5 Hz, 3H). MS (ESI⁺): [M+H]⁺ 336.

### (Z)-2′-(2-(2-(3,5-dimethyl-1H-pyrrol-2-yl)methylene)-3-methoxy-2H-pyrrol-5-yl)-1′,5′,6′,7′-tetrahydrospiro[cyclopropane-1,4′-indole] (Compound 23)

### Synthesis of 1′-tosyl-1′,5′,6′,7′-tetrahydrospiro[cyclopropane-1,4′-indole] (Compound 20)

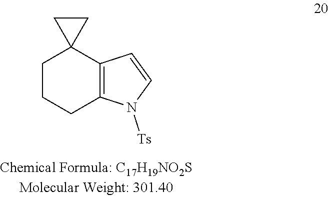 [SMILES: Cc1ccc(S(=O)(=O)n2ccc3c2CCCC32CC2)cc1]

To dry DCM (6.7 mL) at 0° C., was added Et₂Zn (1N solution in hexane, 6.68 mL, 6.68 mmol, 2 equiv). TFA (2M solution DCM, 3.34 mL, 6.68 mmol, 2 equiv) was added dropwise and the resulting solution was stirred at 0° C. for 20 min. CH₂I₂ (2M solution in DCM, 3.34 mL, 6.68 mmol, 2 equiv) was added and the resulting solution was stirred at 0° C. for 20 min. A solution of alkene 4 (960 mg, 3.34 mmol) in dry DCM (5 mL) was added and the resulting orange solution was stirred at 0° C. for 30 min. Sat aq. NH₄Cl was added and the layers were separated. The aqueous phase was extracted with DCM. The combined organic extracts were washed (brine), dried (Na₂SO₄), filtered and concentrated under reduced pressure. The residue was purified by Flash Chromatography (cHex/EA=100/0 to 80/20) to afford 296 mg (29%) of compound 20 as a white solid. ¹HNMR (CDCl₃, 500 MHz): δ 7.70 (m, 2H), 7.32 (m, 2H), 7.14 (dt, J=3.4&1.0 Hz, 1H), (d, J=3.5 Hz, 1H), 1.52 (m, 2H), 0.75-0.71 (m, 2H), 0.67-0.63 (m, 2H).

### Synthesis of 1′,5′,6′,7′-tetrahydrospiro[cyclopropane-1,4′-indole] (Compound 21)

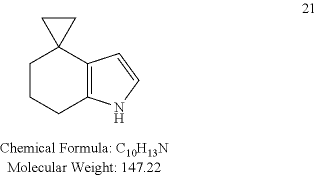 [SMILES: c1cc2c([nH]1)CCCC21CC1]

A suspension of compound 20 (290 mg, 0.962 mmol) in THF/MeOH (1/1, 10 mL) and Mg (233 mg, 9.62 mmol, 10 equiv) and NH₄Cl (103 mg, 1.92 mmol, 2 equiv) were added. The reaction mixture was stirred at RT for 5 h before being partitioned between aq. sat. NH₄Cl and EtOAc. The layers were separated and the aqueous phase was extracted with EtOAc. The combined organic extracts were washed (brine), dried (Na₂SO₄), filtered and concentrated under reduced pressure. The residue was purified by Flash Chromatography (cHex/EA=100/0 to 80/20) to afford 117 mg (83%) of compound 21 as a light blue oil. ¹HNMR (CDCl₃, 500 MHz): δ 7.65 (br s, 1H), 6.51 (app t, J=2.7 Hz, 1H), 5.60 (app t, J=2.7 Hz, 1H), 2.58 (t, J=6.3 Hz, 2H), 1.88 (m, 2H), 1.58-1.52 (m, 2H), 0.71-0.66 (m, 2H), 0.60-0.55 (m, 2H).

### Synthesis of 3-methoxy-5-(1′,5′,6′,7′-tetrahydrospiro[cyclopropane-1,4′-indol]-2′-yl)-1H-pyrrole-2-carbaldehyde (Compound 22)

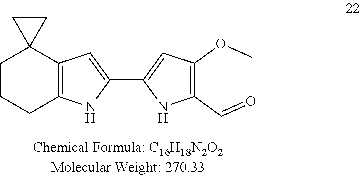 [SMILES: COc1cc(-c2cc3c([nH]2)CCCC32CC2)[nH]c1C=O]

To a solution of compound 21 (115 mg, 0.781 mmol) in hexane (2.5 mL) at RT, were added [Ir(COD)OMe]₂ (10.4 mg, 0.0156 mmol, 0.02 equiv), 4,4 ‘-Di-tert-butyl-2,2’-dipyridyl (8.4 mg, mmol, 0.04 equiv) and B₂pin₂ (119 mg, 0.469 mmol, 0.6 equiv). The reaction mixture was stirred at RT for 2 h. The reaction mixture was concentrated under reduced pressure until ca. 1 mL hexane left. Degassed dioxane/H₂O (9/1, 4 mL), Pd(PPh₃)₄ (63 mg, 0.055 mmol, 0.07 equiv), Na₂CO₃ (248 mg, 2.34 mmol, 3 equiv) and (E)-1-(5-bromo-3-methoxy-2H-pyrrol-2-ylidene)-N,N-dimethylmethanamine (152 mg, 0.586 mmol, 0.75 equiv) were added and the reaction mixture was stirred at 80° C. for 5 h. After cooling down to RT, EtOAc (6 mL), water (6 mL) and 2N HCl (1.2 mL to reach pH=7) were added and the suspension was stirred for 0.5 h. The resulting slurry was filtered over fritted glass and the filter cake was washed with water (3*2 mL) and EtOAc (3*2 mL). The yellow cake was dried under vacuum for 1 h to afford 98 mg (62%) of compound 22 as a yellow solid. ¹HNMR (DMSO-d₆, 500 MHz): δ 11.14 (br s, 1H), 10.77 (br s, 1H), 9.25 (s, 1H), 6.22 (s, 1H), 6.16 (s, 1H), 3.82 (s, 3H), 2.63 (t, J=6.2 Hz, 2H), 1.85 (br s, 2H), 1.55 (br s, 2H), 0.65 (br s, 4H).

### Synthesis of (Z)-2′-(2-((3,5-dimethyl-1H-pyrrol-2-yl)methylene)-3-methoxy-2H-pyrrol-5-yl)-1′,5′,6′,7′-tetrahydrospiro[cyclopropane-1,4′-indole] (Compound 23)

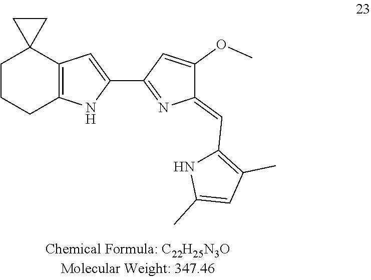 [SMILES: COC1=CC(c2cc3c([nH]2)CCCC32CC2)=N/C1=C\c1[nH]c(C)cc1C]

To a suspension of compound 22 (50 mg, 0.18 mmol) in MeOH (0.75 mL) at RT, were added 2,4-dimethylpyrrole (35 mg, 0.37 mmol, 2.0 equiv) and HCl (1.25N solution in MeOH, 0.22 mL, 0.28 mmol, 1.5 equiv). The reaction mixture was stirred at RT for 24 h. The reaction mixture was filtered over fritted glass and the grey filter cake was washed with MeOH (3*1.5 mL). The filter cake was collected and suspended in MeOH (1 mL). Saturated NH₄OH solution in water was added (0.2 mL) and the resulting suspension was stirred at RT for 15 min. It was filtered over fritted glass and the filter cake was washed with MeOH (3*2 mL), collected and dried under vacuum to afford 26 mg (40%) of compound 23 as a red solid. ¹HNMR (CDCl₃, 500 MHz): δ 6.82 (s, 1H), 6.06 (s, 1H), 6.00 (s, 1H), 5.66 (s, 1H), 3.97 (s, 3H), 2.26 (br s, 2H), 2.19 (s, 3H), 1.85-1.65 (m, 5H), 1.57-1.45 (m, 2H), 0.76-0.71 (m, 2H), 0.67-0.62 (m, 2H).

### Example B—Biology

### Example B1—Antiviral Effect

The antiviral effect of the compounds of the invention have been tested on A549 cell lines infected with H1N1 (influenza A/New Caledonia/20/99). IC50 are reported in the following Table 1. The results show that the compounds of the present invention present an improved antiviral effect compare to the reference compound Obatoclax.

TABLE 1

| Compound | IC50 (nM) |
|---|---|
| Obatoclax | 91 |
| Compound 3 | 56 |
| Compound 7 | 75 |
| Compound 19 | 91 |
| Compound 23 | 77 |

### Materials and Methods

### Cells and Virus

The A549 human lung epithelial cell line and the Madin-Darby canine kidney (MDCK) cells (ECACC) were grown in DMEM media (GibCo, 41966052) supplemented with 100 Um′ penicillin/streptomycin (GibCo, 15140130) and 10% fetal calf serum (PAN, 3302-P221126) at 37° C. and 5% CO2.

The epidemic A/H1N1/New Caledonia/2006 were propagated in MDCK cells in DMEM supplemented with 1 mg·ml-1 modified trypsin TPCK (Sigma, T3053) in absence of FCS. Virus stocks were titrated by standard plaque assay on MDCK cells using an agar overlay medium.

### Molecules

All the molecules were solubilized in DMSO at a stock concentration of 10 mM. 9 serial 2-fold dilutions of test compounds were prepared in DMEM, starting at 1 mM.

### Virus Infection

A549 cells were washed twice with D-PBS 1× (GibCo, 14190). Molecules were added at indicated concentrations. Cells were then infected with H1N1 (MOI 0.1) in DMEM supplemented with 0.2 mg·ml-1 trypsin TPCK (infection medium) and incubated for 48 h in infections medium at 37° C. and 5% CO2.

### Titer Measure by Neuraminidase Activity

Influenza virus neuraminidase is able to cleave the methyl-umbelliferyl-N-acetyl neuraminic acid (4-MUNANA, Sigma, M8639) modifying its emission wavelength in a dose-dependent manner. In 96-black plate (Corning, 3631), 25 ml infection supernatants were diluted in 25 ml D-PBS 1× containing calcium and magnesium (GibCo, 14040) and 50 ml of 20 mM 4-MUNANA.

After 1 h incubation at 37° C., 100 ml of glycine 0.1M 25% ethanol pH10.7 was added. Measures were done with TECAN infinite M1000 instrument at 365 nm excitation wavelength and 450 nm emission wavelength.

1-14. (canceled)

15. A compound of formula (I): 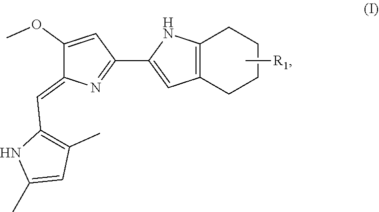
wherein R₁ represents a hydrogen, a (C₁-C₆)alkyl group, or a spiro-connected (C₃-C₆)cycloalkyl group, and stereoisomers and pharmaceutical salts thereof.

16. The compound according to claim 15, wherein R₁ represents a hydrogen, a methyl group, or a spiro-connected cyclopropyl group.

17. The compound according to claim 15, wherein said compound is of formula (I′): 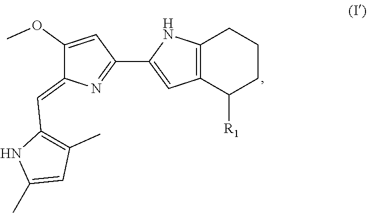
in which R₁ is a hydrogen, a (C₁-C₆)alkyl group, or a spiro-connected (C₃-C₆)cycloalkyl group.

18. The compound according to claim 15, wherein said compound is selected from the group consisting of:
(Z)-2-(2-((3,5-dimethyl-1H-pyrrol-2-yl)methylene)-3-methoxy-2H-pyrrol-5-yl)-4,5,6,7-tetrahydro-1H-indole (Compound 3);
(Z)-2-(2-((3,5-dimethyl-1H-pyrrol-2-yl)methylene)-3-methoxy-2H-pyrrol-5-yl)-4-methyl-4,5,6,7-tetrahydro-1H-indole (Compound 7);
(Z)-2-(2-((3,5-dimethyl-1H-pyrrol-2-yl)methylene)-3-methoxy-2H-pyrrol-5-yl)-5-methyl-4,5,6,7-tetrahydro-1H-indole (Compound 19); and
(Z)-2′-(2-(2-(3,5-dimethyl-1H-pyrrol-2-yl)methylene)-3-methoxy-2H-pyrrol-5-yl)-1′,5′,6′,7′-tetrahydrospiro[cyclopropane-1,4′-indole] (Compound 23).

19. A pharmaceutical composition comprising a compound according to claim 15 and a pharmaceutically acceptable excipient.

20. A method of treating a viral infection comprising administering a compound according to claim 15 or a pharmaceutical composition comprising said compound and a pharmaceutically acceptable excipient to a subject in need of treatment.

21. The method according to claim 20, wherein the viral infection is an infection by a virus selected from the group consisting of Heunggongvirae, Monodnaviria, Orthornavirae, Pararnavirae, Shotokuvirae, Artverviricota, Cossaviricota, Duplornaviricota, Kitrinoviricota, Negarnaviricota, Peploviricota and Pisuviricota.

22. The method according to claim 20, wherein the viral infection is an infection by a virus of a family selected from the group consisting of Coronaviridae, Flaviviridae, Hepadnaviridae, Herpesviridae, Orthomyxoviridae, Papillomaviridae, Paramyxoviridae, Picornaviridae, Polyomaviridae, Reoviridae, Retroviridae, Rhabdoviridae, and Togaviridae.

23. The method according to claim 22, wherein the virus is from Orthornavirae, Negarnaviricota, or Orthomyxoviridae.

24. The method according to claim 23, wherein the virus is selected from the group consisting of Influenza virus A, Influenza virus B, Influenza virus C, Isavirus, Thogotovirus and Quaranjavirus.

25. The method according to claim 24, wherein the Influenza virus A is selected from subtypes H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7.

26. A method of treating cancer comprising administering a compound according to claim 15 or a pharmaceutical composition comprising said compound and a pharmaceutically acceptable excipient to a subject in need of treatment.

27. The method according to claim 26, wherein the cancer is selected from the group consisting of a breast cancer, a lung cancer, a melanoma, a colorectal cancer, an astrocytoma cancer, a liver cancer, leukemia, lymphoma, a gastric cancer, a head and neck cancer, a cervical cancer, a pancreatic cancer, an ovarian cancer, myeloma and myelodysplastic syndromes.

28. The method according to claim 27, wherein the lung cancer is small cell lung cancer or non-small cell lung cancer.

29. The method according to claim 27, wherein said leukemia is acute myeloid leukemia or chronic lymphocytic leukemia.

30. The method according to claim 27, wherein said lymphoma is follicular lymphoma, non-Hodgkin's lymphoma, Hodgkin's lymphoma, or recurrent adult diffuse large cell lymphoma.

31. The method according to claim 30, wherein said myelodysplastic syndromes are myelofibrosis and mastocytosis.

32. The method according to claim 27, wherein said myeloma is multiple myeloma.
